(19) United States
(12) Patent Application Publication
(10) Pub. No.: US 2026/0137606 A1
(43) Pub. Date: May 21, 2026

(54) DENTIFRICE COMPOSITION

(71) Applicant: HALEON UK IP LIMITED, Weybridge, Surrey (GB)

(72) Inventors: Shaila ALI, Weybridge, Surrey (GB); Piotr PIOTROWSKI, Weybridge, Surrey (GB)

(21) Appl. No.: 19/116,427

(22) PCT Filed: Oct. 5, 2023
(86) PCT No.: PCT/EP2023/077660

§ 371 Date: Mar. 28, 2025

(30) Foreign Application Priority Data
Oct. 6, 2022 (GB) 2214700.3

(51) Int. Cl. A61K 8/21; A61K 8/25; A61K 8/34; A61K 8/46; A61K 8/73; A61Q 11/00

(52) U.S. Cl. CPC A61K 8/21; A61K 8/25; A61K 8/345; A61K 8/463; A61K 8/731; A61Q 11/00; A61K 2800/5922

(57) ABSTRACT

The invention relates to a single-phase dentifrice composition that is capable of stabilising two mutually incompatible oral care actives.

### FIELD OF THE INVENTION

The invention relates to oral care compositions and in particular dentifrice (preferably toothpaste) formulations. The invention relates to a stable dentifrice composition with two actives with different stability requirements.

### BACKGROUND TO THE INVENTION

Toothpaste formulations with potassium nitrate can provide daily sensitivity protection.

Potassium is an ingredient that is clinically proven to reduce dentin hypersensitivity. See: Jeandot J et al. Clinic (French) 2007; 28:379-384; Leight R S et al. J Clin Dent 2008; 19:147-153; Nagata T et al. J Clin Periodontol 1994; 21:217-221; Silverman G. Compend Contin Educ Dent 1985; 6:131-133,136 and Silverman G et al. Am J Dent 1994; 7 (1): 9-12.

Potassium nitrate formulations build protection over time to help desensitize nerves in tooth pulp.

Potassium ions can travel into exposed dentin tubules from the tooth surface to reach internal nerves. It is the build-up of potassium ions over time helps desensitize nerves in the tooth pulp, making them irresponsive to sensitivity stimuli (e.g. cold water, hot coffee, etc.)

Stannous fluoride is also an active ingredient often used in toothpastes to achieve, amongst other benefits, a reduction in dentin hypersensitivity.

Stannous fluoride has a different mode of action to achieve a reduction in dentin sensitivity that potassium nitrate. It is believed stannous fluoride accelerates the occlusion of exposed dentin tubules.

There has long been a desire to utilise the combination of these two actives in a single-phase toothpaste, for superior sensitivity control.

However, this has never been satisfactorily achieved. The primary reason for this being that the two actives have very specific, and opposite, formulation needs. Potassium nitrate is an inorganic salt and as such needs, typically, a formula that is highly polar and most preferably aqueous to be stable. Stannous fluoride, conversely, is usually unstable in an (neutral) aqueous environment. Stannous fluoride is commercialised in predominantly non-aqueous formulations (such as in the Sensodyne® range of toothpastes) to prevent unacceptable levels of decomposition on the shelf.

Previously toothpaste compositions employing both these actives at therapeutically useful levels have required the use of two component pastes. Often in striped toothpastes. One formula optimised for one active and the other phase for the other active. These are kept separate in the tube until dispensation onto the brush.

It is the object of the present invention to solve this problem and provide an effective, stable, single-phase toothpaste that provides these benefits.

### STATEMENTS OF INVENTION

The invention comprises a single phase, non-aqueous dentifrice composition comprising; glycerol, potassium nitrate, stannous fluoride, a fumed silica and at least one hydroxyethylcellulose polymer.

In a further embodiment of the invention the hydroxyethylcellulose polymer has a formula weight average molecular weight (Da) of between about 500,000 and about 1000000 Da, preferably between about 600,000 and about 900,0000 Da, more preferably between about 650,000 and about 800,000 Da and most preferably between about 700,000 and about 750,000 Da.

In a further embodiment the hydroxyethylcellulose polymer comprises between about 0.05% and about 1% by weight, more preferably between about 0.1% and about 0.5% by weight and most preferably between about 0.2% and about 0.4% by weight.

In a further embodiment, the fumed silica is present between about 1% and 6% by weight, more preferably between about 2% and about 4% by weight.

In a further embodiment the glycerol comprises at least about 50% by weight of the composition, more preferably at least about 60% by weight and most preferably at least 70% by weight.

In a further embodiment the potassium nitrate comprises between about 1% and about 8% by weight more preferably between about 3% and about 7% and most preferably around about 5% by weight of the total formulation.

In a further embodiment the stannous fluoride comprises between about 0.1% and about 1% by weight of the formulation, preferably between about 0.3% and about 0.7% by weight and most preferably between about 0.4% and about 0.6% by weight.

In a further embodiment the composition further comprises one or more surfactants, wherein the one or more surfactants comprises between about 0.5% and about 5% by weight, preferably between 1.0 and 4.0% by weight and most preferably between about 2.0% and about 3.0% by weight.

In a further embodiment the one or more surfactants comprises an anionic surfactant.

In a further embodiment the surfactant is sodium lauryl sulfate (SLS).

In a further embodiment the composition further comprises a thickening gum between about 0.05% and about 5% by weight of the composition, more preferably between about 0.075 and about 1% by weight and most preferably between about 0.1 and about 0.5% by weight.

In a further embodiment the thickening gum comprises carrageenan.

In a further embodiment of the invention the composition comprises one or more silicas, wherein the total silica content is between about 1% and about 20% by weight, preferably between about 5% and about 15% by weight.

In a further embodiment the composition further comprises one or more stain removing ingredients between about 0.5% and about 10% by weight, preferably between about 2.5% and about 7.5% and most preferably about 5% by weight.

In a further embodiment the composition further comprises one or more additional ingredients selected from the categories of flavourings, sweeteners, colourants and preservatives.

### DETAILED DESCRIPTION

The applicants have surprisingly found a non-aqueous single phase dentifrice formulation that is stable to therapeutic levels of both potassium nitrate and stannous fluoride.

A dentifrice is a powder, paste or liquid composition for cleaning teeth. The most common form of dentifrice is a toothpaste.

The formulas provide excellent shelf-life stability and consumer acceptability.

Stannous fluoride is well known to cause staining issues and can cause yellowing in toothpastes. This is believed to be the result of chemical decomposition and interactions, but the exact mechanism is not well understood. The formulas of the invention offer remarkable resistance to this degradation effect, with no discolouration seen even in extended storage.

Non-aqueous for the present invention means less than about 5% by weight total water content, preferably less than about 2.5% by weight and most preferably less than about 1% by weight of water.

The dentifrice formulations of the present invention are based on glycerol as solvent in combination with at least one hydroxyethylcellulose polymer as a thickener and a fumed silica.

Glycerol (also called glycerine or glycerin) is a simple polyol compound. It is a colourless, odourless, viscous liquid that is sweet-tasting and non-toxic.

The dentifrice compositions of the present invention comprise at least about 50% by weight glycerol, preferably at least about 60% by weight glycerol and most preferably at least about 70% by weight glycerol.

It is well known that commercially available glycerin may contain between about 0.1% to about 2.0% by weight of water which is in association with the glycerin. Typically this amount is <0.5% for example between about 0.1% and about 0.5% by weight of the glycerin. This small amount of water is bound to the glycerin and is therefore not available to the other ingredients. The skilled person would still consider a composition containing glycerin as being non-aqueous. The solvent should in any case be as anhydrous as possible.

Hydroxyethyl cellulose is a gelling and thickening agent derived from the natural polymer cellulose.

Cellulose is an organic compound with the formula (C₆H₁₀O₅)ₙ, a polysaccharide consisting of a linear chain of several hundred to many thousands of B (1→4) linked D-glucose units. Cellulose is an important structural component of the primary cell wall of green plants, many forms of algae and the oomycetes. Cellulose is the most abundant organic polymer on Earth.

Hydroxy ethylcellulose polymers are widely used in cosmetics, cleaning solutions, and other household products.

The hydroxyethylcellulose polymers are commercially available products and come in a wide variety of different specifications. For the purposes of the present invention a hydroxyethylcellulose polymer with a weight average molecular weight (Daltons or Da) of between 500,000 and 1,000,000 Da is preferred.

More preferably a hydroxyethylcellulose polymer with a weight average molecular weight of between 600,000 and 900,000 Da and most preferably between 700,000 and 800,000 Da.

A particularly preferred hydroxyethylcellulose polymer for use in the formulations of the present invention is Natrosol™ 250 M pharm grade which is made by Ashland. This polymer is about 720,000 Da.

Preferable the hydroxyethyl cellulose polymer comprises between 0.05 and 1% by weight, more preferably between 0.1% and 0.5% by weight and most preferably between 0.2 and 0.4% by weight of the total dentifrice composition.

The ratio of weight ration of glycerol to hydroxethyl polymer in the dentifrice compositions of the present invention is from about 2000:1 to about 60:1, preferably about 1500:1 to about 120:1 and most preferably from 750:1 to about 200:1, most preferably between from about 500:1 to about 250:1.

It is particularly preferable for the dentifrice compositions of the present invention to comprise a weight ratio of glycerol to hydroxyethyl cellulose of about 300:1. This provides a single-phase base formulation that is able to stabilise a therapeutic level of both potassium nitrate and stannous fluoride in combination.

The compositions of the invention require a fumed silica to achieve stability.

Fumed silica (also known as pyrogenic silica because it is produced in a flame) consists of microscopic droplets of amorphous silica fused into branched, chainlike, three-dimensional secondary particles which then agglomerate into tertiary particles. The resulting powder has an extremely low bulk density and high surface area.

Preferably the fumed silica of the present invention has a specific surface area of at least 150 m²/g, and more preferably at least 200 m²/g

A particularly preferred fumed silica is Aerosil® 200 as sold by the Evonik chemical company. Aerosil® 200 is a hydrophilic fumed silica with a specific surface area of 200 m²/g. Another preferred fumed silica is Aerosil® 300 as sold by the Evonik chemical company. Aerosil® 300 is a hydrophilic fumed silica with a specific surface area of 300 m²/g.

Other suitable sources of fumed silica include: Cabot Corporation (Cab-O-Sil®), Wacker Chemie (HDK®), Dow Corning, Heraeus (Zandosil®), Tokuyama Corporation (Reolosil®), OCI (Konasil®), Orisil (Orisil) and Xunyuchem (XYSIL®)

Preferably the fumed silica is present between about 1% and about 6% by weight of the formulation, more preferably between about 2% and about 4% by weight.

Potassium nitrate may be used in a wide range of different concentrations.

Potassium nitrate may be found in the dentifrices of the present invention between about 1% about and 8% by weight, preferably between about 3% and about 7% by weight.

Therapeutic clinical effects of potassium nitrate in dentifrice compositions are typically seen at about 5% by weight. This is a particularly preferred amount for the present invention.

Stannous fluoride may also be tolerated and be stable in a wide range of different concentrations with the compositions of the present invention.

The amount of stannous fluoride that may be used in commercial toothpastes is highly regulated by law and different in different legal jurisdictions, but the formulations of the present invention are stable to all of these and in concentrations much higher that legally allowable also.

Stannous fluoride may be used between about 0.1% and about 3% by weight, preferably between about 0.2% and about 2.0% by weight, more preferably between about 0.3% and about 1.0% by weight and most preferably about 0.4% and 0.7% by weight.

A particularly preferred level of Stannous fluoride for commercial toothpastes is 0.454% or 1100 ppm. Other preferred levels are 1450 ppm and 900 ppm.

An optional ingredient that can be used to impart further stability is an aliphatic polyether. Preferred polyethers include polyethylene glycol (PEG) and polypropylene glycol (PPG) or combinations thereof.

If included in the formulations of the present invention, the PEG or PPG is preferably included between 1% and 25% by weight of the formulation, preferably between 5% and 15% by weight.

The formulations of the present invention may comprise one or more further thickening agents and one or more additional solvent(s). Optionally, a dentally acceptable abrasive may be included in the non-aqueous carrier.

Advantageously, a thickening agent may be present in the formulation to adjust the rheology to that of a conventional tube-based dentifrice.

Suitably the thickening agent comprises an additional polymer. One possible polymer type are carboxyvinyl polymers such as a carbomer. A carbomer comprises synthetic high molecular weight cross-linked polymers of acrylic acid. The polymer chains formed of repeating units of acrylic acid may be cross-linked with, for example: allyl sucrose to provide a carbomer available commercially in one form as Carbopol™ 934; ethers of pentaerythritol to provide a carbomer available commercially in one form as Carbopol™ 974; or with divinyl glycol, available commercially in one form as Noveon™ AA-1 Carbopol™ polymers are manufactured by B.F. Goodrich Company. In one embodiment the carboxyvinyl polymer comprises Carbopol™ 974. The carboxyvinyl polymer may be present in the range of from about 0.1 to about 7.5% by weight of the non-aqueous composition. In one embodiment the carboxyvinyl polymer is present in an amount from about 0.3 to about 1.0% by weight of the composition.

Suitably and either additionally or alternatively a composition according to the invention may comprise at least one inorganic thickening agent such as a thickening silica. Suitably, the thickening agent is a thickening silica, for example, a colloidal hydrated silica, available commercially for example as Sident 22S or Syloid 244FP.

The compositions of the present invention may comprise a mixture of two or more different grades of silica with differing thickening and abrasive properties.

In one embodiment the total amount of thickening and/or abrasive silica is present in the range of from about 0 to about 15%, suitably from about 5.0 to about 15.0% by weight of the non-aqueous composition.

The toothpaste compositions of the present invention preferably contain at least one surfactant.

The at least one surfactant may be any surfactant that are known for use in oral care compositions.

A particularly preferred class of surfactant are anionic surfactants and more particularly those of the C₁₀₋₂₀ alkyl sulphate surfactant class.

Alkyl sulphate surfactants of use in the invention have the following structural formula:

R¹OSO₃M

R¹ represents a fatty alcohol moiety and M represents sodium, potassium, ammonium or triethanolamine. Fatty alcohols having carbon chain lengths of from about 10 to about 20, including those derived from coconut, palm oil and tall oil. In one embodiment, the fatty alcohol is lauryl alcohol. In one embodiment, a sodium salt is used. In one embodiment the alkyl sulphate is sodium lauryl sulphate.

The alkyl sulphate surfactant may be present in an amount from about 0.1% to about 10% of the non-aqueous composition. In one embodiment the alkyl sulphate surfactant may be present in an amount from about 0.1 to about 5% by weight of the single phase non-aqueous composition. In one embodiment the alkyl sulphate surfactant is present in an amount from about 0.5% to about 2.0% by weight of the non-aqueous composition.

The most preferred surfactant of these is sodium lauryl sulphate.

Sodium laurylsulfate specifically may be present between about 0.1% and about 3% by weight, preferably between about 0.5% and about 2.5% and most preferably between about 1 and about 2% by weight.

Another preferred surfactant for use in the composition according to the invention is a taurate surfactant. Taurate surfactants useful in the present invention are salts of fatty acid amides of N-methyl taurine. They conform generally to the structural formula:

RC(O)N(CH₃)CH₂CH₂SO₃M

Where RC(O)— represents a fatty acid radical and M represents sodium, potassium, ammonium or triethanolamine. Fatty acids having carbon chain lengths of from 10 to 20, including those derived from coconut, palm and tall oil are used. In one embodiment the fatty acid is derived from coconut. In one embodiment, sodium salts are used. In one embodiment the taurate is sodium methyl cocyl taurate. This taurate surfactant is sold under the trademark by Adinol® CT by Croda.

The taurate surfactant may be present in an amount from about 0.1% to about 10% of the non-aqueous composition. In one embodiment the taurate surfactant is present in an amount from about 0.1% to about 5.0% by weight of the non-aqueous composition. In one embodiment the taurate surfactant is present in an amount from about 0.5% to about 2.0% by weight of the non-aqueous composition.

Another surfactant class suitable for use in the compositions of the invention belongs to the class of compounds known as betaines. Structurally, betaine compounds contain an anionic functional group such as a carboxylate functional group and a cationic functional group such as quaternary nitrogen functional group separated by a methylene moiety. They include n-alkyl betaines such as cetyl betaine and behenyl betaine, and n-alkylamido betaines such as cocoamidopropyl betaine. In one embodiment the betaine is cocoamidopropyl betaine, commercially available under the trade name Tego Betain. Suitably the betaine is present in an amount ranging from about 0.05% to about 4% by weight of the non-aqueous composition, for example from about 0.2% to about 2.0% by weight of the non-aqueous composition.

A composition according to the invention may also comprise a surfactant system. The surfactant system consists of a first surfactant and at least a single second surfactant. In certain embodiments the surfactant system consists of a first surfactant and a second surfactant wherein the second surfactant consists of a mixture of two or more surfactants.

In certain embodiments, the surfactant system consists of a mixture of at least two surfactants from the list of betaines, taurates and alkyl sulfates and mixtures thereof.

It is preferable that formulations of the present invention include at least one anticalculus ingredient or reagent.

Polyphosphates are known to help retard calculus formation and are examples of anticalculus agents suitable for use in the dentifrice compositions of the present invention. A polyphosphate is generally understood to consist of two or more phosphate groups arranged primarily in a linear configuration, although some cyclic derivatives may be present. Polyphosphates of use in the invention include pyrophosphates, polyphosphates having three or more polyphosphate groups such as sodium tripolyphosphate, and polyphosphates having four or more polyphosphate groups such as tetrapolyphosphate and hexametaphosphate among others.

Polyphosphates may be used between about 1% and about 12% by weight of the composition, preferably between about 2% and about 10% by weight and more preferably between about 3% and about 7% by weight.

A particularly preferred polyphosphate for use in the present invention is sodium tropolyphosphate.

Other chemical anticalculus ingredients may be used as required.

Other examples of such ingredients include sodium pyrophosphate and sodium hexametaphosphate.

The toothpaste compositions of the present invention may also comprise bioactive glass components.

An example of such an additive is a bioactive glass of the type disclosed in WO96/10985, WO 97/27158 and WO 99/13852. In an aqueous environment such a bioactive glass releases ions causing a significant increase in pH which can adversely affect the stability (especially upon long-term storage) of any excipients contained within the dentifrice. Formulating a bioactive silica-based glass in the non-aqueous dentifrice of the present invention prevents the release of ions within the dentifrice thereby controlling pH and increasing long-term storage stability of the dentifrice.

In one embodiment the bioactive glass for use in the invention has a composition consisting of about 45% by weight silicon dioxide, about 24.5% by weight sodium oxide, about 6% by weight phosphorus oxide, and about 24.5% by weight calcium oxide. One such bioactive glass is available commercially under the trade name, NovaMin®, also known as 45S5 Bioglass®.

The bioactive glass may be present in an amount ranging from about 1% to about 20% by weight of the non-aqueous composition. In one embodiment, the bioactive glass is present in an amount from about 1% to about 15% by weight of the non-aqueous composition. In an alternative embodiment, the bioactive glass in the non-aqueous composition is present in an amount from about 1% to about 10% by weight of the non-aqueous composition. In a further alternative embodiment, the bioactive glass is present in an amount from about 2% to about 8% by weight of the non-aqueous composition.

A dentally acceptable abrasive may optionally be added to the non-aqueous compositions of the present invention. Advantageously, the presence or absence of a dentally acceptable abrasive as well as the amount of such abrasive may be used to selectively control the abrasive level of the dentifrice composition made of the invention. By way of example, and if present, the bioactive glass may provide an acceptable level of abrasive for the non-aqueous composition depending upon the ultimate use.

By further way of example, a desired amount of dentally acceptable abrasive may be added to increase the abrasive level of the overall non-aqueous composition.

Suitable abrasives for use in the non-aqueous composition of the invention include, for example, amorphous, gelled, precipitated or fumed silica, zinc orthophosphate, sodium bicarbonate (baking soda), plastic particles, alumina, hydrated alumina, calcium carbonate, calcium pyrophosphate, insoluble metaphosphates or mixtures thereof.

The silica abrasive may be a natural amorphous silica, for instance diatomaceous earth; or a synthetic amorphous silica such as a precipitated silica. By way of example, silica abrasives include those marketed under the following trade names Zeodent®, Sident®, Sorbosil® or Tixosil® by Huber, Degussa, Ineos and Rhodia respectively.

Suitably a silica abrasive is present in an amount up to about 25% by weight of the total composition, for example from about 2% to about 20% by weight for example from about 5% to about 15% by weight of the total composition.

The formulations of the present invention comprise stannous fluoride. Additional sources of fluoride may also be included to adjust the fluoride to the required level. Suitable sources of fluoride ions for use in the compositions of the present invention include an alkali metal fluoride such as sodium fluoride, an alkali metal monofluorophosphate such a sodium monofluorophosphate, stannous fluoride, or an amine fluoride in an amount to provide from 25 ppm to 3500 ppm of fluoride ions, preferably from 100 to 1500 ppm.

Compositions of the invention may further comprise an anti-erosion agent, for example a polymeric mineral surface-active agent as described in WO 04/054529 (Procter & Gamble).

Compositions of the present invention will contain additional formulating agents such as flavouring agents, sweetening agents, opacifying or colouring agents and preservatives, selected from those conventionally used in an oral hygiene composition art for such purposes. In general, these additional formulating agents may be used in a minor amount or proportion of the overall formulation. By way of example, such components are usually present in from about 0.001 to about 5% by weight of the non-aqueous composition.

The dentifrice composition typically has a viscosity suitable for application to the oral cavity. The viscosity will vary depending on the type of dentifrice composition made and the ultimate use thereof. One of skill in the art can readily prepare compositions with suitable viscosities for use in the oral cavity from the teachings provided herein.

The compositions according to the present invention may be prepared by admixing the ingredients in the appropriate relative amounts in any order that is convenient.

The invention is further illustrated by the following Examples.

### Examples

TABLE 1

| Ingredients | Example 1 | Example 2 | Example 3 |
|---|---|---|---|
| Glycerol | 74.2460 | 74.1739 | 75.3960 |
| Hydrated Dental Type Silica - | 6.0000 | 6.0000 | 6.0000 |
| Zeodent ® 124 | | | |
| Sodium Tripolyphosphate | 5.0000 | 5.0000 | 5.0000 |
| Potassium Nitrate | 5.0000 | 5.0000 | 5.0000 |
| Silica - Fumed Silica - Aerosil ® 200 | 3.2000 | 3.2000 | 3.2000 |
| Silicon Dioxide (Medium/High | 0.7500 | 0.7500 | |
| Thickening Silica) | | | |
| Precipitated Silica - Sident ® 22S | | | |
| Sodium Lauryl Sulfate | 2.0000 | 2.0000 | 2.0000 |
| Flavour | 1.2000 | 1.2000 | 1.2000 |
| Colourant | 1.0000 | 1.0000 | 1.0000 |
| Sweetener | 0.5000 | 0.5000 | 0.5000 |
| Stannous Fluoride | 0.4540 | 0.4540 | 0.4540 |
| Hydroxyethyl cellulose polymer | 0.2500 | 0.2500 | 0.2500 |
| (Natrosol ® 250M from Ashland) | | | |
| Carrageenan | 0.4000 | 0.4000 | |
| Sodium Fluoride | | 0.0721 | |
| Total | 100.0000 | 100.0000 | 100.0000 |

TABLE 2

| Ingredients | Example 4 | Example 5 | Example 6 | Example 7 |
|---|---|---|---|---|
| Glycerol | 73.7960 | 74.5460 | 72.0960 | 72.1960 |
| Hydrated Dental Type Silica - | 6.0000 | 6.0000 | 6.0000 | 6.000 |
| Zeodent ® 124 | | | | |
| Sodium Tripolyphosphate | 5.0000 | 5.0000 | 5.0000 | 5.000 |
| Potassium Nitrate | 5.0000 | 5.0000 | 5.0000 | 5.000 |
| Silica - Fumed Silica - Aerosil ® 200 | 3.2000 | 3.2000 | 3.2000 | 3.2000 |
| Silicon Dioxide (Medium/High | 1.5000 | 0.7500 | 3.2000 | 3.2000 |
| Thickening Silica) | | | | |
| Precipitated Silica - Sident ® 22S | | | | |
| Sodium Lauryl Sulfate | 2.0000 | 2.0000 | 2.0000 | 2.000 |
| Flavour | 1.2000 | 1.2000 | 1.2000 | 1.2000 |
| Titanium Dioxide | 1.0000 | 1.0000 | 1.0000 | 1.0000 |
| Sweetener | 0.5000 | 0.5000 | 0.5000 | 0.5000 |
| Stannous Fluoride | 0.4540 | 0.4540 | 0.4540 | 0.4540 |
| Hydroxyethyl cellulose | 0.2500 | 0.2500 | 0.2500 | 0.2500 |
| (Natrosol ® 250M from Ashland) | | | | |
| Carrageenan | 0.1000 | 0.1000 | 0.1000 | |
| Total | 100.0000 | 100.0000 | 100.0000 | 100.0000 |

TABLE 3

| Ingredients | Example 8 | Example 9 | Example 10 | Example11 |
|---|---|---|---|---|
| Glycerol | 73.3960 | 73.2960 | 76.1960 | 76.0960 |
| Hydrated Dental Type Silica - | 2.0000 | 2.0000 | 2.0000 | 2.0000 |
| Zeodent ® 124 | | | | |
| Sodium Tripolyphosphate | 5.0000 | 5.0000 | 5.0000 | 5.0000 |
| Potassium Nitrate | 5.0000 | 5.0000 | 5.0000 | 5.0000 |
| Silica - Fumed Silica - Aerosil ® 200 | 3.2000 | 3.2000 | 3.2000 | 3.2000 |
| Silica Dental Type (Medium Thickening | 6.0000 | 6.0000 | | |
| Silica) - Zeodent ® 153B | | | | |
| Silicon Dioxide (Medium/High | | | 3.2000 | 3.2000 |
| Thickening Silica) - Precipitated | | | | |
| Silica - Sident ® 22S | | | | |
| Sodium Lauryl Sulfate | 2.0000 | 2.0000 | 2.0000 | 2.0000 |
| Flavour | 1.2000 | 1.2000 | 1.2000 | 1.2000 |
| Titanium Dioxide | 1.0000 | 1.0000 | 1.0000 | 1.0000 |
| Sweetener | 0.5000 | 0.5000 | 0.5000 | 0.5000 |
| Stannous Fluoride | 0.4540 | 0.4540 | 0.4540 | 0.4540 |
| Hydroxyethyl cellulose | 0.2500 | 0.2500 | 0.2500 | 0.2500 |
| (Natrosol ® 250M from Ashland) | | | | |
| Carrageenan | | 0.1000 | | 0.1000 |
| Total | 100.0000 | 100.0000 | 100.0000 | 100.0000 |

TABLE 4

| Ingredients | Example 12 | Example 13 | Example 14 |
|---|---|---|---|
| Glycerol | 64.4440 | 59.4440 | 54.4440 |
| Propylene Glycol | 5.0000 | 10.0000 | 15.0000 |
| Hydrated Dental Type Silica - Zeodent ® 124 | 6.0000 | 6.0000 | 6.0000 |
| Sodium Tripolyphosphate | 5.0000 | 5.0000 | 5.0000 |
| Potassium Nitrate | 5.0000 | 5.0000 | 5.0000 |
| Silica - Fumed Silica - Aerosil ® 200 | 4.0000 | 4.0000 | 4.0000 |
| Silica, Dental Type (Low Abrasive Silica) | 4.0000 | 4.0000 | 4.0000 |
| Silicon Dioxide (Medium/High Thickening Silica) | 1.5000 | 1.5000 | 1.5000 |
| Precipitated Silica - Sident ® 22S | | | |
| Sodium Lauryl Sulfate | 2.0000 | 2.0000 | 2.0000 |
| Flavouring/colouring | 1.0020 | 1.0020 | 1.0020 |
| Sweetener | 0.3000 | 0.3000 | 0.3000 |
| Stannous Fluoride | 0.4540 | 0.4540 | 0.4540 |
| Hydroxyethyl cellulose (Natrosol ® 250M from Ashland) | 1.0000 | 1.0000 | 1.0000 |
| Carrageenan | 0.3000 | 0.3000 | 0.3000 |
| Total | 100.0000 | 100.0000 | 100.0000 |

All examples exhibited required stability levels in accelerated tests (3M at 40° C. and 75% relative humidity) in terms of active levels and visible paste stability (no syneresis or obvious discolouration).

1. A single phase, non-aqueous dentifrice composition comprising; glycerol, potassium nitrate, stannous fluoride, a fumed silica and at least one hydroxyethylcellulose polymer.

2. The single phase, non-aqueous dentifrice composition of claim 1, wherein the hydroxyethylcellulose polymer has a formula weight average molecular weight (Da) of between 500,000 and 1000000 Da, preferably between 600,000 and 900,0000 Da, more preferably between 650,000 and 800,000 Da and most preferably between 700,000 and 750,000 Da.

3. The single phase, non-aqueous dentifrice composition of claim 1, wherein the hydroxyethylcellulose polymer comprises between 0.05% and 1% by weight, more preferably between 0.1% and 0.5% by weight and most preferably between 0.2% and 0.4% by weight.

4. The dentifrice of claim 1, wherein the fumed silica is present between 1% and 6% by weight, more preferably between 2% and 4% by weight.

5. The single phase, non-aqueous dentifrice composition of claim 1, wherein the glycerol comprises at least 50% by weight of the composition, more preferably at least 60% by weight and most preferably at least 70% by weight.

6. The single phase, non-aqueous dentifrice composition of claim 1, wherein the potassium nitrate comprises between 1% and 8% by weight more preferably between 3% and 7% and most preferably around 5% by weight of the total formulation.

7. The single phase, non-aqueous dentifrice composition of claim 1, wherein the stannous fluoride comprises between 0.1% and 1% by weight of the formulation, preferably between 0.3% and 0.7% by weight and most preferably between 0.4% and 0.6% by weight.

8. The single phase, non-aqueous dentifrice composition of claim 1, wherein the composition further comprises at least one surfactant, wherein the at least one surfactant comprises between 0.5% and 5% by weight, preferably between 1.0% and 4.0% by weight and most preferably between 2.0% and 3.0% by weight.

9. The single phase, non-aqueous dentifrice composition of claim 8, wherein the at least one surfactant comprises an anionic surfactant.

10. The single phase, non-aqueous dentifrice composition of claim 8, wherein the one or more surfactants is sodium lauryl sulfate (SLS).

11. The single phase, non-aqueous dentifrice composition of claim 1, wherein the composition further comprises a thickening gum between 0.05% and 5% by weight of the composition, more preferably between 0.075% and 1% by weight and most preferably between 0.1% and 0.5% by weight.

12. The single phase, non-aqueous dentifrice composition of claim 11, wherein in the thickening gum comprises carrageenan.

13. The single phase, non-aqueous dentifrice composition of claim 1, wherein the composition further comprises one or more additional silicas, wherein the total silica content is between 1% and 20% by weight, preferably between 5% and 15% by weight.

14. The single phase, non-aqueous dentifrice composition of claim 1, wherein the composition additionally comprises one or more additional sources of fluoride.

15. The dentifrice composition of claim 1, wherein the composition further comprises one or more additional ingredients selected from the categories of flavourings, sweeteners, colourants, whitening agents, pH adjusting agents, rheology modifiers, antimicrobial agents, preservatives and mixtures thereof.
